(19) United States
(12) Patent Application Publication
(10) Pub. No.: US 2006/0269967 A1
(43) Pub. Date: Nov. 30, 2006

(54) Novel diagnosis & treatment tools for cancer using the NRIP antibody and RNA interference

(76) Inventors: Show-Li Chen, Taipei City (TW); Yeou-Ping Tsao, Taipei City (TW); Tzung-Chieh Tsai, Taichung City (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 11/138,335

(22) Filed: May 27, 2005

(51) Int. Cl. G01N 33/567; G01N 33/574; C07H 21/04; C12P 21/06; C07K 14/72; C07K 16/30

(52) U.S. Cl. 435/7.2; 435/7.23; 435/69.1; 435/320.1; 435/325; 530/350; 530/388.22; 536/23.5

(57) ABSTRACT

The invention provides an isolated nucleic acid encoding NRIP (Nuclear Receptor Interaction Protein) gene that highly expressed in tumorigenic development of human neoplasms in cervical cancer. The invention provides an antibody against the NRIP protein, and methods of diagnosing the NRIP related cancer. The invention also features RNA interferences of the NRIP gene, and the methods of treatment for the NRIP related cancer using the RNA interferences.

### BACKGROUND OF THE INVETION

1. Field of the invention

The present invention relates to cancer, more particularly to a cancer related gene, and to the diagnosis and treatment of cancer using the antibody and the RNA interference of the cancer related gene.

2. Description of the prior art

The role of steroids and steroid receptors in the occurrence of disease and as targets for disease prevention is widely recognized and is currently an active area of research (Wiseman et al., Biochem. Soc. Trans., 2001, 29, 205-209). In the past decade, several coactivators have been cloned and characterized that associate with steroid receptors and enhance their ability to transactivate target genes (Horwitz et al., Mol. Endocrinol., 1996, 10, 1167-1177; and Recent Prog. Horm. Res., 1997, 52, 1-502). Given that these coactivators have intrinsic activation functions, these factors most likely enhance assembly of basal transcription factors into a stable PIC (preinitiation complex), resulting in increased transcription initiation rates of RNA polymerase II (Jenster et al., Proc. Natl. Acad. Sci. U.S.A, 1997, 94, 7879-7884). Surprisingly, the coactivators AIB1 (Amplified In Breast Cancer-1) (Anzick et al., Science, 1997, 277, 965-968) and PBP/PPARBP (peroxisome proliferator-activated receptor binding protein) (Zhu et al., Proc. Natl. Acad. Sci. U.S.A, 1999, 96, 10848-10853) have been found that was amplified in both breast and ovarian cancers. These two proteins interact with estrogen receptors in a ligand-dependent fashion, and functions to enhance estrogen-dependent transcription. It was suggested that the aberrant expression of these genes maybe contribute to the development of steroid hormone dependent cancers.

Cervical cancer is currently believed to arise in association with high-risk type of human papillomavirus (HPV) infection. Nevertheless, virus infection and viral gene expression emerge as necessary but obviously not sufficient factors for cancer induction. Additional modifications of host cell genes appear to be required for malignant progression of infected cells (Moodley et al., Int. J. Gynecol. Cancer, 2003, 13, 103-110). Mostly, regulation of viral transcription transpires mainly within the control region of the genome. A large number of cellular transcription factors binding to specific binding sites in the long control region (LCR) of HPVs have been identified (Bernard et al., Arch. Dermatol., 1994, 130, 210-215; Gloss et al., J. Virol., 1989, 63, 1142-1152; and Chong et al., J. Virol., 1991, 65, 5933-5943). Moreover, at least three glucocorticoid-responsive element (GRE) within the long control region (LCR) of the human papilloma virus type 16 (HPV-16) has been designated (Mittal et al., Obstet. Gynecol., 1993, 81, 5-12). It has been shown both in vitro and in vivo that steroid hormones (e.g. dexamethasone, 17 beta-estradiol, progesterone) enhance the transcription of the HPV genome (E6/E7 gene) via modulation their upstream regulatory region (URR) (von Knebel et al., Proc. Natl. Acad. Sci. U.S.A, 1991, 88, 1411-1415; Chong et al., J. Virol., 1991, 65, 5933-5943; and Yuan et al., Cancer Invest, 1999, 17, 19-29). The transforming potentials of E6 and E7 from the high-risk viruses interact strongly with the tumor suppressors p53 and Rb (Werness et al., Science, 1990, 248, 76-79; Dyson et al., Science, 1989, 243, 934-937; and Munger et al., EMBO J., 1989, 8, 4099-4105), respectively. Consequently inactivate and decrease the activity of the tumor suppressors and impair the control of cell cycle checkpoint. This supports their role in the maintenance of the proliferative phenotype of cervical carcinoma cells. However, to date there is little known about the molecular evidence from human studies explaining the role of HPV and steroid hormones in the genesis of cervical cancer. Therefore, it will be an important issue to investigate whether steroid hormones and nuclear receptors coregulators are involved in the regulation the HPV genome transcription and the progression of cervical cancer.

### SUMMARY OF THE INVENTION

The present invention is based on the identification of a human gene that highly expressed in tumorigenic development of human neoplasms in cervical cancer. This gene has been designated the “NRIP” (Nuclear Receptor Interaction Protein) gene. Thus, the invention features 29 nucleic acid sequences encoding the potential antigenic peptides of NRIP protein, an antibody against the NRIP protein, the analysis of the gene expression pattern of NRIP, and methods of diagnosing the NRIP related cancer. In addition, the invention features 7 RNA interferences of the NRIP gene, an in vitro method of inhibiting proliferation of cancer cells, and the methods of treatment for the NRIP related cancer using the RNA interferences. More specifically, the invention features an isolated DNA containing a nucleic acid sequence encoding a polypeptide of SEQ ID No: 3. The DNA includes the nucleic acid sequence of SEQ ID No: 2. The invention features 29 nucleic acid sequences of SEQ ID No: 4 to SEQ ID No: 32 that encoding the potential antigenic peptides of NRIP protein.

The invention provides an antibody capable of specifically binding to the polypeptide of SEQ ID No: 3. The antibody is a monoclonal antibody or a polyclonal antibody. Furthermore, the antibody could be detected by labeling with a detectable marker. The detectable marker can be a radioactive label or a colorimetric, or a luminescent, or a fluorescent marker.

In another aspect, the invention provides a method of diagnosis. The method involves (a) providing a test sample form patient; (b) determining the level of an antigen of a protein that having the amino acid sequence of SEQ ID No: 3 in the sample with the antibody of the invention; and (c) comparing the level of the antigen in the sample to a reference value representing a known disease or health status, whereby any elevated levels of the antigen are indicative of the presence, susceptibility to, or progression of, the cancer in the patient.

Also featured by the invention are 7 RNA interferences for the nucleic acid sequence of SEQ ID No: 2. The target sequences of the 7 RNA interferences are the nucleic acid sequences of SEQ ID No: 33 to SEQ ID No: 39. The invention further featured mutants of the target sequences of the RNA interferences. The target sequences of the RNA interferences could be mutated by substituting alternative nucleic acids for the natural nucleic acids of the sequences.

Another aspect of the invention is a method of inhibiting proliferation of a cancer cell. The method involves administering to a subject a therapeutic RNA interference of the invention in a sufficient amount to decrease the proliferation of the cancer cell. The cancer cell can be, for example, a cervical cancer cell. Alternatively, the cancer cell can be in a mammal.

These features and advantages of the present invention will be fully understood and appreciated from the following detailed description of the accompanying Drawings.

### BRIEF DESCRIPTION OF THE DRAWINGS

FIG. 1 depicts the cDNA (SEQ ID No: 2) sequence and corresponding deduced amino acid sequence (SEQ ID No: 3) of the present invention. The complete nucleotide and deduced amino acid sequence of human NRIP is shown and numbered on the left. The open reading frame of NRIP encodes 860 amino acids. WD40 repeat domains and NLS motif are boxed and underlined respectively (FIG. 1A). Sequence data has been deposited in the GenBank™ database with accession number AY766164. The schematic diagram of NRIP is shown in FIG. 1B. There are seven WD40 domains (squared) and one NLS (solid boxed) in the NRIP protein.

FIG. 2 demonstrates the specificity of the NRIP antibody by immunoblotting. 293T cells were transfected with pcDNA3.1-HisC-NRIP expression plasmid or pcDNA3.1-HisC control vector. Forty-eight hours after transfection, the cell extracts were immunoprecipitated with rabbit polyclonal NRIP antibody and immunobloted with the same antibody (FIG. 2A). Or the cell extracts were immunoprecipitated with anti-Xpress epitope antibody and immunobloted with the same antibody (FIG. 2B).

FIG. 3 illustrates the sublocalization of NRIP in nucleus of 293T cells. Cells were transiently transfected with pEGFP-NRIP plasmid. Forty-eight hours after transfection, cells were fixed with 4% paraformaldehyde and stained with DAPI. Fluorescent EGFP-NRIP was visualized with a ZEISS Axiovert 100M inverted confocal laser microscope. As shown in FIG. 3A, green signal (white arrow) represents the localization of NRIP fusion protein. FIG. 3B shows the phase-contrast image of transfected 293T cells. The blue signal (white arrow) in FIG. 3C represents DAPI-stained nucleus.

FIG. 4 shows the results of immunofluorescence staining for NRIP protein in HeLa cells. HeLa cells were seeded into 8-well chamber slides and fixed with acetone/methanol (1:1, v/v), permeabilizied with saponin, and blocked with normal goat serum. Thereafter, incubation with polyclonal rabbit anti-NRIP antibody in moist chamber for 2 hours. A secondary antibody solution containing goat anti-rabbit IgG conjugated to Texas Red (1:250, v/v) dilutions, Molecular Probes was added to the chamber slides and incubated at room temperature for 1 hour in a moist chamber. After extensive washes, chamber slides were mounted on glass slides with Fluoromount (DAKO) and visualized with a ZEISS Axiovert 100M inverted confocal laser microscope. FIG. 4A shows the negative control of the immunofluorescence assay. FIG. 4B shows the location of NRIP in HeLa cells (white arrows).

FIG. 5 shows the results of immunohistochemical staining of NRIP protein in normal cervix (FIG. 5A), low-grade squamous intraepithelial lesion (LSIL) (FIG. 5B), high-grade SILs (FIG. 5C), squamous cervical carcinoma (FIG. 5D), invasive cervical cancer (FIG. 5E). All photographs were taken with 400× magnification.

FIG. 6 illustrates the RNAi-mediated silencing of exogenous NRIP gene expression. Results showed the RNAi-3 (SEQ ID No: 35) construct could efficiently diminish the exogenous EGFP-NRIP fusion protein expression both in 293T (upper panel) and C33A cells (lower panel).

FIG. 7 illustrates the RNAi-mediated silencing of exogenous NRIP gene expression. Cells were transfected with empty vector (pSuper), RNAi-3, pEGFP-NRIP (wt) or pEGFP-NRIP (mt) constructs as indicated. Lane 1: NRIP+pSuper vector; lane 2: NRIP+RNAi-3; lane 3: mutant NRIP+pSuper vector; lane 4: mutant NRIP+RNAi-3; lane 5: mock. Western bolt analysis shows that RNAi-3 (SEQ ID No: 35) had the capability to inhibit NRIP protein expression (FIG. 7A lane 2, and FIG. 7B lane 2) but not the NRIP mutant both in 293T (FIG. 7A lane 4), and C33A (FIG. 7B lane 4).

FIG. 8 illustrates that RNAi-3 can efficiently inhibit the exogenous NRIP gene expression by fluorescence assay. 293T cell line was transfected with wild type (pEGFP-NRIP) and empty vector (pSuper) (FIG. 8A), or wild type (pEGFP-NRIP) and RNAi-3 (FIG. 8B), or mutant NRIP plasmid (pEGFP-NRIP(mt)) and empty vector (pSUPER) (FIG. 8C), or mutant NRIP plasmid (pEGFP-NRIP(mt)) and RNAi-3 (FIG. 8D). The diminished green color of EGFP-NRIP was only shown in the presence of RNAi-3 and pEGFP-NRIP (wild type) (FIG. 8B), but not in the control vector (pSuper) with pEGFP-NRIP (wild type) (FIG. 8A) or the control plasmid with pEGFP-NRIP(mt) (FIG. 8C) or RNAi-3 plus pEGFP-NRIP(mt) (FIG. 8D).

FIG. 9 illustrates that RNAi-3 (SEQ ID No: 35) can reduce endogenous NRIP gene expression in a dose-dependent manner by RT-PCR (reverse transcription polymerase chain reaction) assay. The expected NRIP product was 1427 bp; β-actin is shown as an RNA loading control.

FIG. 10 illustrates that RNAi-3-mediated silencing of endogenous NRIP gene expression resulted in decreased 293T and C33A cell growth.

### DETAILED DESCRIPTION OF THE PREFERRED EMBODIMENT

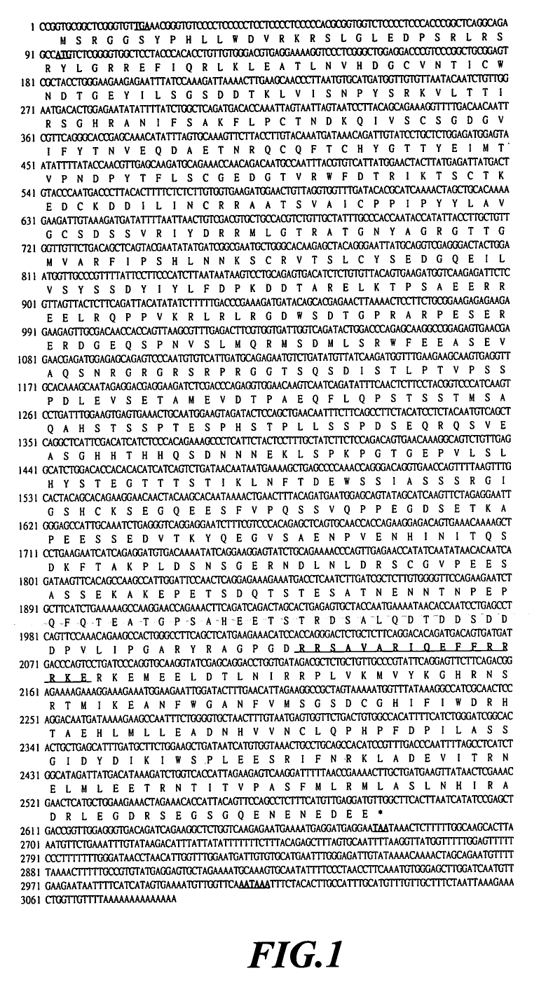

The inventors have isolated a novel gene from human HeLa MATCHMAKER cDNA library using C-terminal domain of androgen receptor (AR, amino acids 595-918) (SEQ ID No: 1) as the bait to carry out yeast-two hybrid screening and named nuclear receptor interaction protein (NRIP) (SEQ ID No: 2). The amino acid sequence (SEQ ID No: 3) analysis of NRIP showed there are seven WD40 domains and one nuclear localization signal (NLS) (FIG. 1). NLS sequence implied that NRIP was a nuclear protein that was confirmed by fluorescence microscopy experiment. Concurrently, the inventors obtained the consistent staining pattern by using polyclonal NRIP antibody in immunofluorescence assay. WD40 domains indicated protein-protein interactions, in vitro and in vivo pull-down assays illustrated that NRIP could interact with either AR or glucocorticoid receptor (GR). Transient transfection and luciferase activity assays demonstrated that when inspected the native promoter of mouse mammary tumor virus (MMTV), NRIP could be regarded as a global transcriptional coactivator of steroid receptors (AR and GR) in ligand-dependent and-non-tissue-specific manner. Moreover, while the inquiry to HPV-16 promoter, NRIP functioned as a selective cofactor of GR-driven transcription (not for AR) in ligand-dependent and was also a tissue-specific coactivator (Tsai et al., J. Biol. Chem., 2005, 280, 20000-20009). To further clarify the function of NRIP, siRNA (small interfering) -mediated NRIP gene silencing in C33A and 293T cells was conducted. The inventors found RNAi-3-targeted NRIP sequence (5′-GATGATACAGCACGAGAAC-3′) (SEQ ID No: 35) that could efficiently and specifically knockdown the endogenous and exogenous NRIP gene and significantly diminished cell proliferation function. Therefore, these data illustrate that the novel gene-NRIP may have dual functions either generally or selectively enhancing transcriptional activity of nuclear receptors in distinct promoter contexts and circumstances.

### EXAMPLE 1

### Materials and Methods

Yeast Two-Hybrid Screen. A pACT2-HeLa MATCHMAKER cDNA library (Clontec) that consists of the GAL4 activation domain (aa 768-881) (SEQ ID No: 41) fused with a human HeLa cDNA library was transformed into CG-1945 yeast strain (Clontec), along with a plasmid pAS2-1-AR595-918 containing GAL4 DBD (aa 1-147) (SEQ ID No: 42) fused with the C-terminal domain of androgen receptor (AR, amino acids 595-918) (SEQ ID No: 1). Approximately 5×10⁶ yeast transformants were screened and selected on synthetic dropout (SD, Difico) medium lacking leucine, tryptophan and histidine in the presence of 25 mM 3-amino-1,2,4-triazole (3-AT, Sigma) and 10 nM dihydrotestosterone (DHT, Sigma). Colonies were tested for LacZ reporter gene activity in a β-Gal filter assay. Plasmid DNAs from positive clones were recovered from yeast, amplified in Escherichia coli, and confirmed by sequencing.

5′-RACE-PCR. 5′-RACE-PCR was used to obtain the remaining 5′ end sequence of the above isolated NRIP gene. The PCR amplification was performed using human HeLa Marathon-ready cDNA (Clontec) as a template. The first amplification was performed using the adaptor primer 1 (AP1) (SEQ ID No: 43) and the gene-specific primer (GSP) 5′-GAGGTCATTTCTTTCTCCTGAGTTGGA-3′ (SEQ ID No: 44) for 28 cycles followed by a final elongation of 10 min at 72° C. Each cycle consisted of 15 s at 94° C., 60 s at 58° C., and 2 min at 72° C.; 1 μl of PCR product was used as a template for the second amplification with the adaptor primer 2 (AP2) (SEQ ID No: 45) and the nested gene-specific primer (NGSP) 5′-ACTGGTTCACCTGTCCCTGGTTTGG-3′ (SEQ ID No: 46) for 28 cycles, using the same conditions as those used for the first amplification. Thereafter the PCR product was cloned into pGEM-T vector (Promega), and sequenced.

Plasmid Constructions. The full-length NRIP was cloned in the mammalian expression vector pcDNA3.1-HisC (Invitrogen) and named pcDNA3.1-HisC-NRIP, containing N-terminal Xpress and histidine epitope tags. The plasmid pEGFP-NRIP was generated by tagging EGFP at the 5′ end of NRIP gene. Mutant NRIP was made by following two PCR-based approaches as described by Mikaelian and Sergeant (Mikaelian, I. and Sergeant, A., Nucleic Acids Res., 1992, 20, 376). Wild-type NRIP was used as a template and the primers sequences are as follows:

(SEQ ID No: 47)

5′-TGCGAATTCATGTCTCGGGGTGGCTCCTACCCACAC-3′
(primer 1);

(SEQ ID No: 48)

5′-GGTGAATTCTTATTCCTCATCCTCATTTTCATTCTCTTG-3′
(primer 2);

(SEQ ID No: 49)

5′-GACCCGAAAGACGACACGGCCCGGGAGCTGAAAACTCCT-3′
(mutagenic primer 3);

(SEQ ID No: 50)

5′-AGGAGTTTTCAGCTCCCGGGCCGTGTCGTCTTTCGGGTC-3′
(mutagenic primer 4).

The mutagenic primer 3 and 4 contained silent mutations (underlined) corresponding to the RNAi3-targeted position (Table 2). Primer 1 and mutagenic primer 4 were used as a pair in one reaction; and mutagenic primer 3 and primer 2 were used in a separate reaction in the first round of PCR. Amplified products were loaded on 1% agarose gel and purified. In the second round of PCR, 20 to 50 ng of each purified fragment were mixed as a template and added to primer 1 and 2 containing EcoRI restriction cutting sites for PCR as described previously (Mikaelian, I. and Sergeant, A., Nucleic Acids Res., 1992, 20, 376). The obtained mutant NRIP fragment was inserted into the pEGFP-C2 vector and named pEGFP-NRIP (mt).

RT-PCR Analysis. For Reverse Transcription Polymerase Chain Reaction (RT-PCR), the total RNA (20 μg) from each sample was reverse-transcribed using M-MLV Reverse Transcriptase (Life Technologies, Inc.). One microliter of cDNA was amplified by PCR using the Expand High Fidelity PCR System (Roche Applied Science). The following forward and reverse primers were used:

(SEQ ID No: 51)

5′-ATGTCTCGGGGTGGCTCCTACCCACAC-3′ (NRIP-F);

(SEQ ID No: 52)

5′-ACTGGTTCACCTGTCCCTGGTTTGG-3′ (NRIP-R);

(SEQ ID No: 53)

5′-ACCTTCAACACCCCAGCCATG-3′ (β-actin-F);

(SEQ ID No: 54)

5′-CTGGAAGAGTGCCTCAGGGCA-3′ (β-actin-R).

These primers amplify respectively 1427 bp of the N-terminal region of NRIP, and 414-bp of the β-actin fragment which was used to relative amounts of RNA and determine among samples.

Western Blot Analysis. 293T and C33A cell lines are transfected with an EGFP-tagged NRIP fusion protein expression plasmid (pEGFP-NRIP) and then treated with siRNA (SEQ ID No: 33 or SEQ ID No: 34 or SEQ ID No: 35 or SEQ ID No: 36 or SEQ ID No: 37 or SEQ ID No: 38 or SEQ ID No: 39). Cell lysates of the transfected cells were separated on 7.5% SDS-PAGE and blotted with specific antibody, and detected using an ECL Western Blotting Detection system (Amersham Biosciences).

Fluorescence Microscopy Assay. Human 293T cells on chamber slides (Nunc) were transfected with 10 μg of pEGFP-NRIP using the Fugene 6 transfection reagent (Roche Applied Science) according to the manufacturer's instructions. Forty-eight hours after transfection, cells were fixed with 4% paraformaldehyde and stained with a nuclear counterstaining dye; 0.2 μg/ml DAPI (4, 6-diamidino-2-phenylindole dihydrochloride, blue color, Sigma). Fluorescent GFP-NRIP was monitored by a ZEISS Axiovert 100M inverted confocal laser microscope.

Cell Culture. 293T and C33A cells were maintained in Dulbecco's modified Eagle's Medium (DMEM) supplemented with 10% fetal bovine serum (FBS) and 1% of MEM non-essential amino acids solution (Gibco BRL).

Design and Construction of siRNA. The pSUPER vector-based RNA interference (RNAi) system (Brummelkamp et al., Science, 2002, 296, 550-553) was used in this study. Based on empirical guidelines (Mittal, Nat. Rev. Genet., 2004, 5, 355-365), seven 19-nucleotide stretches within the coding region of NRIP were designed, being about 50% GC-rich, and unique in the human genome (Table 2) (SEQ UD No: 33 to SEQ ID No: 39). The gene specific targeting sequences was subsequently subcloned downstream of the H1-RNA promoter between the BglII and HindIII sites in a pSUPER vector (a kind gift from Dr. Reuven Agami, Netherlands Cancer Institute, Netherlands).

Cell Proliferation Assay. The Cell Titer 96® Aqueous One Solution Cell Proliferation Assay is a quantitative colorimetric method for determining mammalian cell survival and proliferation. Cells were seeded at 10⁵ cells per well into 24-well plates and maintained in the absence of hormone containing medium for 24-h. Cells were then transiently transfected with 2 μg of RNAi-3 (SEQ ID No: 35) plasmid DNA per well using the Fugene 6 or SuperFect transfection reagent. Post-transfection 24-h, cells were treated with or without 10 nM DHT for 0-, 24-, 48-, and 72-h. 100 μl of Cell Titer 96® Aqueous One Solution Reagent (Promega) was added to each well, and the absorbance at 490 nm was read after incubation for 4-h at 37° C. by a Perkin Elmer Lambda 40 UV/VIS Spectrophotometer.

GenBank™ Accession Number. The human NRIP nucleotide and protein sequences have been submitted to the GenBank™ database with accession numbers AY766164 and AAX09330.

### EXAMPLE 2

### Generation and Analysis of NRIP antibody

The antibody generation protocol begins with the synthesis of antigenic peptides based on non-overlapping regions of the target protein. The NRIP antigenic peptides are determined using the method of Kolaskar and Tongaonkar (Kolaskar et al., FEBS Lett., 1990, 276, 172-174). Predictions are based on analysis of data from experimentally determined antigenic sites on proteins has revealed that the hydrophobic residues Cys, Leu and Val, if they occur on the surface of a protein, are more likely to be a part of antigenic sites. Peptides are selected to be as unique as possible to the target protein and to have minimal homology to closest homologues and to other proteins in the human genome. According to the principle, location of the potential antigenic peptides of the NRIP protein has been predicted and comprised with 29 antigenic determinants (Table 1).

TABLE 1
Antigenic Determinants in NRIP Sequence

| | | | |
|---|---|---|---|
| | Start | | End |
| | Posi- | | Posi- |
| Name | tion | Sequence | tion |

| | | | |
|---|---|---|---|
| NRIP-1 | 6 | SYPHLLWD | 13 |
| NRIP-2 | 37 | FIQRLKLEATLNVHDGCVNT | 56 |
| NRIP-3 | 72 | DTKLVISN | 79 |
| NRIP-4 | 81 | YSRKVLT | 87 |
| NRIP-5 | 95 | ANIFSAKFLPC | 105 |
| NRIP-6 | 107 | NDKQIVSCSGDGVIFYT | 123 |
| NRIP-7 | 133 | RQCQFTCHY | 141 |
| NRIP-8 | 154 | PYTFLSC | 160 |
| NRIP-9 | 192 | RAATSVAICPPIPYYLAVGCSDSSVRI | 218 |
| NRIP-10 | 240 | MVARFIPS | 247 |
| NRIP-11 | 250 | NNKSCRVTSLCYS | 262 |
| NRIP-12 | 266 | QEILVSYSSDYIYLF | 280 |
| NRIP-13 | 303 | RQPPVKR | 309 |
| NRIP-14 | 379 | DISTLPTVPSSPDLEV | 394 |
| NRIP-15 | 405 | AEQFLQP | 411 |
| NRIP-16 | 431 | PHSTPLLSS | 439 |
| NRIP-17 | 472 | TGEPVLSLHY | 481 |
| NRIP-18 | 519 | EESFVPQSSVQP | 530 |
| NRIP-19 | 546 | DVTKYQEG | 553 |
| NRIP-20 | 589 | LDRSCGVPE | 597 |
| NRIP-21 | 659 | DDPVLIPG | 666 |
| NRIP-22 | 677 | RSAVARI | 683 |
| NRIP-23 | 704 | IRRPLVKMVYK | 714 |
| NRIP-24 | 738 | SDCGHIFI | 745 |
| NRIP-25 | 750 | TAEHLML | 756 |
| NRIP-26 | 758 | EADNHVVNCLQPHPFDPILASS | 779 |
| NRIP-27 | 781 | IDYDIKIWS | 789 |
| NRIP-28 | 819 | TITVPAS | 825 |
| NRIP-29 | 827 | MLRMLASLNH | 836 |

The 29 predicted antigenic peptides were synthesized by Genemed Synthesis, Inc. The peptides were determined to be >80% pure by HPLC. A portion of each synthetic peptide was coupled to keyhole limpet hemocyanin (KLH), by the carbodiimide method, for immunization purposes. New Zealand White rabbits were immunized with 120 μg of each KLH-NRIP-predicted antigenic peptide in complete Freund's adjuvant administered by multiple subcutaneous injections along the back and proximal limbs. Subsequent boosts (100 μg of KLH-coupled peptide in Freund's incomplete adjuvant) were given subcutaneously every 2 weeks. After five boosts, plasmaphoresis was performed. Sera from the immunized rabbits are tested for binding to the cognate peptides by ELISA (enzyme-linked immunosorbent assay). One of the putative antigenic peptide, NRIP-26 (Table 1) (SEQ ID No: 4), has been successfully elicited a specific NRIP antibody production from the immunized rabbits. Thus, polyclonal antibody directed specifically against NRIP-26 (SEQ ID No: 4) was obtained and used throughout the invention.

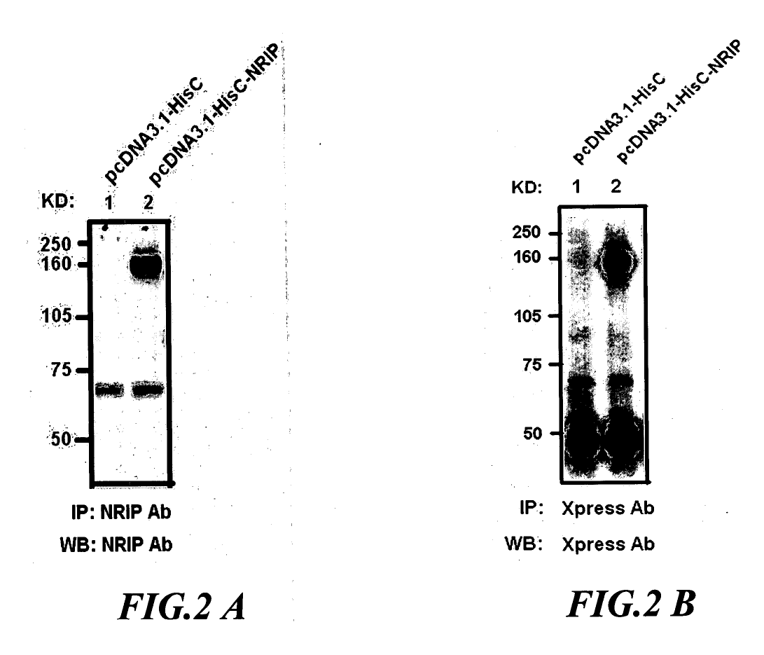

To analyze the quality of the anti-NRIP-26 antibody, in vivo immunoprecipitation assay was conducted. 293T cells were transfected with a plasmid encoding Xpress-tagged NRIP (pcDNA3. 1-HisC-NRIP expression plasmid) or control vector. Forty-eight hours after transfection, the cell extracts were immunoprecipitated with rabbit polyclonal NRIP antibody and immunobloted with the same antibody. The NRIP protein was detected with an apparent molecular mass of 160 kDa (FIG. 2A). The significantly higher mass than predicted (96 kDa) may be due to aberrant electrophoretic mobility imparted by the highly charged amino acid content (40%) of the protein sequence. Concurrently, Xpress-tagged epitope antibody was employed and detected Xpress-tagged fusion protein of NRIP in immunoprecipitation assay (FIG. 2B). These results indicated that the NRIP antibody could specifically against NRIP protein and used throughout all examples of the invention.

### EXAMPLE 3

### The Locations of NRIP in Mammalian Cells

The NRIP gene was originally isolated from the HeLa cDNA library by yeast-two hybrid screening. Sequence analysis of domain architectures in human NRIP are shown that contains seven WD40 domains and one nuclear translocation sequence (NLS). NLS sequence suggested that NRIP may be a nuclear protein. WD40 repeats are conserved sequence motifs of 40 residues with a GH dipeptide 11-24 residues from its N-terminus and the WD dipeptide at its C-terminus and probable contribute to protein-protein interactions (Neer et al., Nature, 1994, 371, 297-300; and Smith et al., Trends Biochem. Sci., 1999, 24, 181-185). WD40 repeats-containing NRIP may play the role for protein-protein interactions.

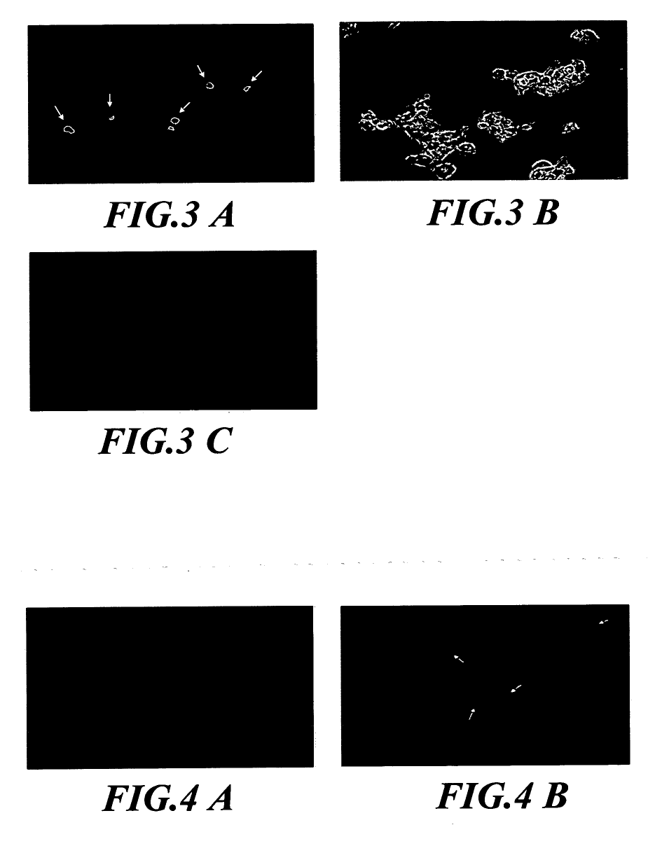

To clarify the location of NRIP in mammalian cells, immunofluorescence assay was conducted. 293T cells were transiently transfected with pEGFP-NRIP plasmid. Forty-eight hours after transfection, cells were fixed with 4% paraformaldehyde and stained with 4′-6-Diamidino-2-phenylindole (DAPI). Fluorescent EGFP-NRIP was visualized with a ZEISS Axiovert 100M inverted confocal laser microscope. As shown in FIG. 3A, green signal (white arrow) represents the localization of NRIP fusion protein. Subcellular compartment of NRIP was restricted to nucleus. FIG. 3B shows the phase-contrast image of transfected 293T cells. The blue signal in FIG. 3C represents DAPI-stained nucleus, and it confirms that the localization of NRIP in nucleus.

To further clarify the location of NRIP in HeLa cells, the NRIP antibody used in the present study detects as demonstrated by immunofluorescence staining assay. Subconfluent cells were seeded on 8-well chamber slides and fixed with acetone/methanol (1:1, v/v), permeabilized with saponin, and blocked with 10% normal goat serum. Incubation with polyclonal rabbit anti-NRIP antibody at 1:300 (v/v) dilutions was carried out at room temperature in a moist chamber for 2-h. A secondary antibody solution containing goat anti-rabbit IgG conjugated to Texas Red (1:250 (v/v) dilutions, Molecular Probes) was added to the chamber slides and incubated at room temperature for 1-h in a moist chamber. After extensive washes, chamber slides were mounted on glass slides with Fluoromount (DAKO) and visualized with a ZEISS Axiovert 100M inverted confocal laser microscope. FIG. 4A shows the negative control of the immunofluorescence assay. FIG. 4B shows that NRIP protein predominantly located in perinuclear and nucleoli (white arrows).

The results of the immunofluorescence assay illustrated that the subcompartments of NRIP protein located in perinuclear and nucleoli. Based on the nuclear localization and NRIP association with AR from yeast-two hybrid assay, we inferred that NRIP might play a role for regulating transcription activity of nuclear receptors.

The eukaryotic nucleus contains a number of domains or subcompartments, which include nucleoli, nuclear Cajal bodies, nuclear speckles, transcription and replication foci, and chromosome territories (Lamond et al., Science, 1998, 280, 547-553). Recently, however, the nucleolus has been implicated in many aspects of cell biology that include functions such as gene silencing, senescence, and cell cycle regulation (Hiscox, Arch. Virol., 2002, 147, 1077-1089). It suggested that the location of NRIP in nucleus may govern the regulations of cell growth and variety physiological events.

### EXAMPLE 4

### NRIP Over-Expresses in Pre-Cancerous and Squamous Cervical Carcinoma (SCC), But Not in Both Normal and Invasive Cervical Regions

The NRIP gene was originally isolated from the HeLa cDNA library using C-terminal domain of androgen receptor (AR, amino acids 595-918) (SEQ ID No: 1) as bait to carry out yeast-two hybrid screening. Cervical cancer is the second leading cause of cancer death of women in worldwide (Rhe et al., Int. J. Cancer, 222001, 93, 44424-429). However, nuclear receptors, such as AR and GR, play an important role in some hormone-responsive tumors (Wiseman, and Duffy, Biochem. Soc. Trans., 2001, 29, 205-209). Therefore, the inventors investigated hormone-related disease, such as cervical and prostate cancers. Before investigating whether NRIP was involved in hormone-related regulation, the inventors investigate the expression pattern of NRIP in clinical cervical cancer specimens by using an immunohistochemistry assays. The tumors were classified into histological subtypes according to the criteria of Reagan et al. (Reagan et al., Lab Invest, 1957, 6, 241-250). These tissue samples were used in the detection of the expression of NRIP protein by immunohistochemistry assays. Initially, an avidin-biotin immunohistochemistry was performed on 4-μm sections from routinely processed paraffin-embedded tissues. The sections were incubated with NRIP antibody (1:100 dilution) for 1-h at room temperature, then incubated with biotinylated anti-rabbit antibody (1:200 dilution; DAKO) for 1-h at room temperature, and then incubated with the avidin-biotin complex (DAKO). The substrate-chromogen, 3% amino-9-ethylcarbazone (DAKO), was added. After extensive washes, slides were mounted on glass slides with Fluoromount (DAKO) and examined with light microscopy (Olympus-CH-2, Japan).

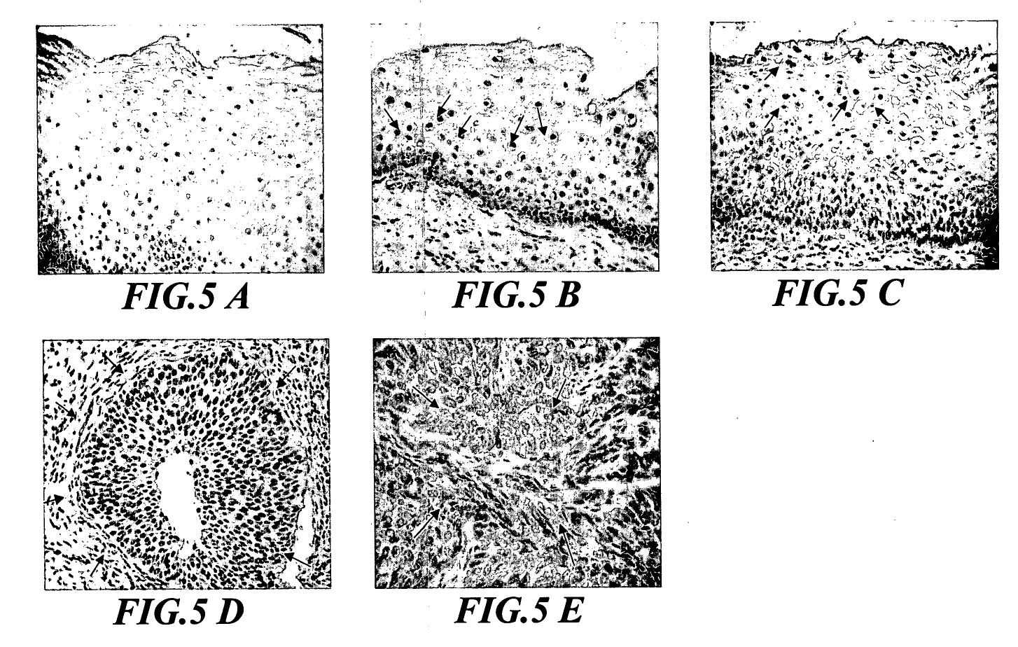

The results of the immunohistochemistry assay depicted in FIG. 5 show that NRIP protein appears to be abundant in both squamous cervical carcinoma (SCC) (FIG. 5D) and pre-cancerous lesion that includes low grade squamous intraepithelial lesion (LSIL) (FIG. 5B) and high grade squamous intraepithelial lesion (HSIL) (FIG. 5C). Moreover, NRIP is predominantly expressed in nuclei of LSIL (FIG. 5B, black arrows) and HSIL (FIG. 5C, black arrows) differentiated epithelial cells of cervical cancer lesions. Human papillomaviruses (HPV) are recognized to play an etiologic role in cervical carcinogenesis and are detectable in almost all pre-invasive and invasive cervical epithelial neoplasms (Shiffman et al., J. Natl. Cancer Inst., 1993, 85, 958-964; Walboomers et al, J. Pathol., 1999, 189, 12-19). Intriguingly, a koilocytosis phenomenon was observed in both LSIL and HSIL stage of histologic samples which may correlate with HPV infection. However, different HPV subtypes have been shown to have different oncogenic potentials and broadly classified into two categories: low-risk or high-risk. Indeed, cervical cancer association with high-risk HPV infection with strains such as HPV-16 and HPV-18 have been verified (Zur, Biochim. Biophys. Acta, 1996, 1288, F55-F78). Integration of high-risk HPV sequences into the cell genome is considered to be an important event in the progression of cervical neoplasia by causing disruption of the E2 gene. Thus, it may result in overexpression of viral E6 and E7 oncogenes that are necessary for immortalization and transformation of cervical keratinocytes (Munger et al., J. Virol., 1989, 63, 4417-4421). In the inventors′ previous research, it indicated that NRIP may enhance AR- and GR-mediated transcriptional activity in HPV-16 promoter (Tsai et al., J. Biol. Chem., 2005, 280, 20000-20009). In addition, the inventors' unpublished data shown that decreasing NRIP gene expression effects on E7 gene expression of HPV-16. Consequently, it inhibited the growth of CaSki cells. Furthermore, NRIP protein is dramatically reduced following they progression to invasive stages of cervical cancer. Therefore, NRIP, also as a useful biomarker of cervical intraepithelial neoplasia (CIN), shows increased immunoexpression with worsening grades of CIN. Taken together, NRIP may regulate HPV gene expression and also involved in the progression of cervical cancer.

### EXAMPLE 5

### Construction and Analysis of NRIP RNA Interference

RNA interference (RNAi) has been proven to be a powerful tool to silence gene expression in a sequence-specific manner. Recent advances in the understanding of RNAi have provided practical tools to knockdown gene expression in mammalian cells, thus make it possible to quickly generate gene knockout models for investigating the functions of NRIP genes on the nuclear receptor transactivation, the pSUPER vector-based small interfering RNA (siRNA) system (Brummelkamp et al., Science, 2002, 296, 550-553) was used in this study.

Based on empirical guidelines (Mittal, Nat. Rev. Genet., 2004, 5, 355-365), seven 19-nucleotide stretches within the coding region of NRIP were designed and they were about 50% GC-rich, and unique in the genome (Table 2) (SEQ ID No: 33 to SEQ ID No: 39). For example, the RNAi-1-specific targeted sequence (SEQ ID No: 33) of NRIP corresponding to nucleotides 580-598 was designed as follows: 5′-GATCCCCGATGGAACTGTTAGGTGGTttcaagagaACCACCTA ACAGTTCCATCTTTTTGGAAA-3′. The gene specific targeting sequence was subsequently subcloned downstream of the H1-RNA promoter between the BglII and HindIII restriction enzyme cutting sites in a pSUPER vector (kindly gift from Dr. Reuven Agami, Netherlands Cancer Institute, Netherlands). RNAi-2,-3,-4,-5, -6, and RNAi-7 (SEQ ID No: 34 to SEQ ID No: 39) were also constructed in a similar manner. Therefore, short 19-nt stem-loop structures of the various siRNAs were designed to correspond to the NRIP nucleotide positions shown in Table 2.

TABLE 2
Target Sequences for NRIP RNAi Constructs

| | | |
|---|---|---|
| Name | Sequence | Position |

| | | |
|---|---|---|
| RNAi-1 | 5′-GATGGAACTGTTAGGTGGT-3′ | 580-598 |
| RNAi-2 | 5′-GATGGTCAAGAGATTCTCG-3′ | 883-901 |
| RNAi-3 | 5′-GATGATACAGCACGAGAAC-3′ | 943-961 |
| RNAi-4 | 5′-GAGTTGCGACAACCACCAG-3′ | 994-1012 |
| RNAi-5 | 5′-CACCAATCCTGAGCCTCAG-3′ | 1965-1983 |
| RNAi-6 | 5′-CTGCCTGCAGCCACATCCG-3′ | 2388-2406 |
| RNAi-7 | 5′-TCATATCCGAGCTGACCGG-3′ | 2598-2616 |

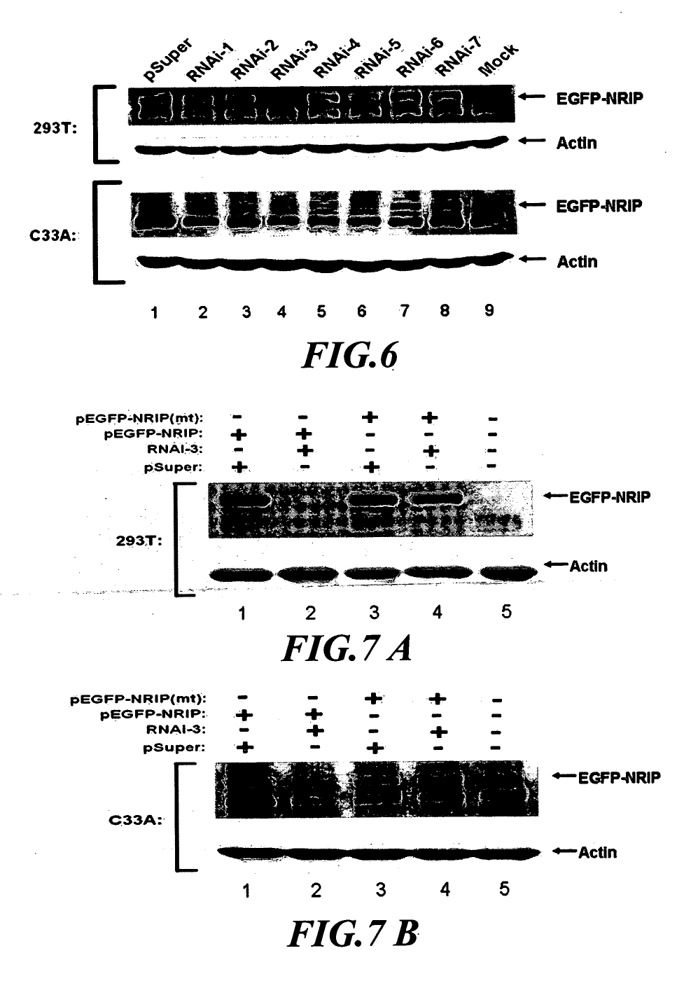

To analysis the effect of these seven siRNA constructs on inhibiting NRIP protein expression, two cell lines, 293T and C33A, were transfected with an EGFP-tagged NRIP fusion protein expression plasmid (pEGFP-NRIP) and then treated with each siRNA construct. Cell lysates were analyzed for expression of EGFP-NRIP fusion protein by Western blot using specific antibodies for GFP (sc-9996, Santa Cruz Biotech.), or actin (MAB-1501, CHEMICON) as an internal control. Results showed the RNAi-3 (SEQ ID No: 35) construct could efficiently diminish the exogenous EGFP-NRIP fusion protein expression both in 293T (FIG. 6, upper panel) and C33A cells (FIG. 6, lower panel).

### EXAMPLE 6

### RNAi-3 Could Knock Down Exogenous NRIP Gene Expression

To further determine whether RNAi-3 construct could specifically knock down exogenous NRIP gene expression, the inventors generated the mutant plasmid pEGFP-NRIP(mt), in which sequence corresponding to RANi-3-targeted-sequence (SEQ ID No: 35) (position 943 to 961) were mutated to 5′-GACGACACGGCCCGGGAGC-3′ (SEQ ID No: 40) (as shown in Table 3). Wild type (pEGFP-NRIP) or mutant NRIP plasmid (pEGFP-NRIP(mt)) were co-transfected with either RNAi-3 or empty vector (pSUPER) into 293T and C33A. Western bolt analysis shows that RNAi-3 (SEQ ID No: 35) had the capability to inhibit NRIP protein expression (FIG. 7A lane 2, and FIG. 7B lane 2) but not the NRIP mutant both in 293T (FIG. 7A lane 4), and C33A (FIG. 7B lane 4).

TABLE 3

| | | |
|---|---|---|
| Name | Sequence | Position |

| | | |
|---|---|---|
| pEGFP-NRIP | 5′-GATGATACAGCACGAGAAC-3′ | 943-961 |
| pEGFP-NRIP (mt) | 5′-GACGACACGGCCCGGGAGC-3′ | 943-961 |

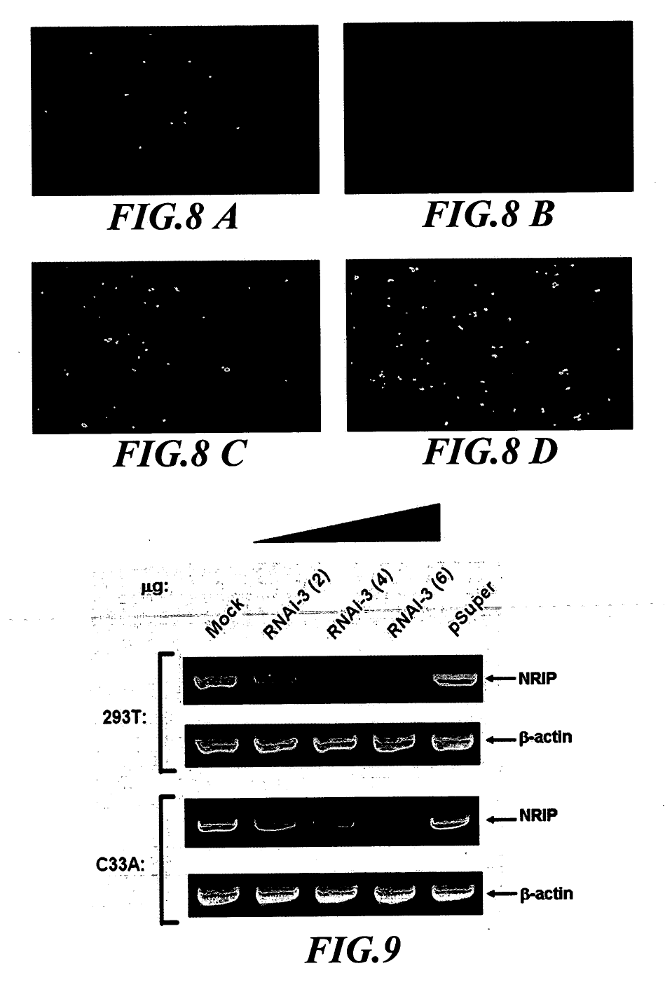

Furthermore, the results of FIG. 7A were also confirmed by fluorescence microscopy (FIG. 8). 293T cell line was transfected with wild type (pEGFP-NRIP) or mutant NRIP plasmid (pEGFP-NRIP(mt)) combined with RNAi-3 or empty vector (pSUPER) respectively. Forty-eight hours after transfection, cells were monitored by ZEISS Axiovert 100M inverted confocal laser microscope. The diminished green color of EGFP-NRIP was only shown in the presence of RNAi-3 and pEGFP-NRIP (wild type) (FIG. 8B), but not in the control vector (pSuper) with pEGFP-NRIP (wild type) (FIG. 8A) or the control plasmid with pEGFP-NRIP(mt) (FIG. 8C) or RNAi-3 plus pEGFP-NRIP(mt) (FIG. 8D). Therefore, RNAi-3 targeted to the 19-nucleotides (5′-GATGATACAGCACGAGAAC-3′) (SEQ ID No: 35) position 943 to 961 of NRIP can efficiently inhibit the exogenous NRIP gene expression.

### EXAMPLE 7

### RNAi-3 has the Capability to Reduce Endogenous NRIP Gene Expression in a Dose-Dependent Manner

Moreover, to investigate whether RNAi-3 (SEQ ID No: 35) could silence endogenous NRIP gene expression, cells (293T and C33A) were transfected with increasing concentrations (2, 4, and 6 μg) of RNAi-3-containing plasmid construct as indicated. The total plasmid amount was adjusted with empty pSUPER vector to 10 μg. Forty-eight hours post-transfection, cells were harvested, and total RNA was isolated by using TRIzol reagent (Gibco BRL), and endogenous NRIP gene expression was assayed by RT-PCR (reverse transcription polymerase chain reaction) analysis. As shown in FIG. 9, the expected NRIP product was 1427 bp; β-actin is shown as an RNA loading control. RT-PCR analysis shows that RNAi-3 (SEQ ID No: 35) has the capability to reduce endogenous NRIP gene expression in a dose-dependent manner in 293T (FIG. 9, upper panel) and C33A cells (FIG. 9, lower panel).

Taken the results of example 5, 6, and 7 together, the designed sequence of RNAi-3 could target NRIP gene and diminished its gene expression.

### EXAMPLE 8

### RNAi-3-Mediated Silencing of Endogenous NRIP Gene Expression Results in Decreased 293T and C33A Cell Growth

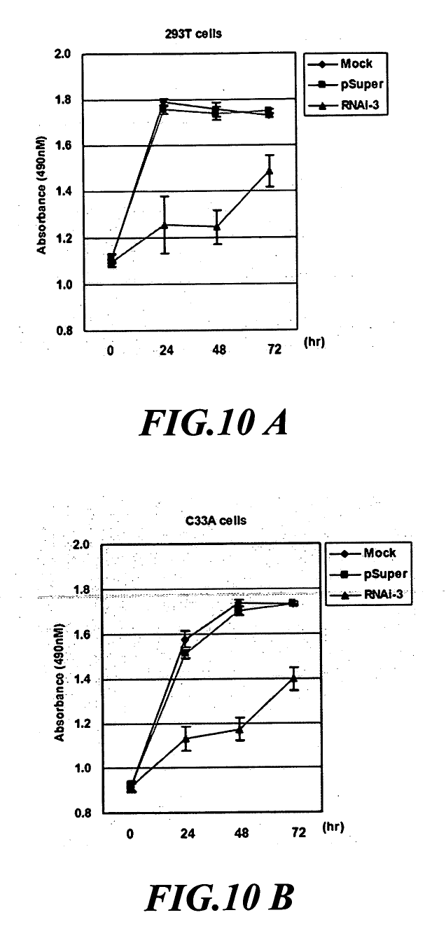

Thereafter, in order to determine the biological effect of RNAi-3-inhibiting NRIP gene expression, the inventors measured the cell proliferation rates of RNAi-3 transfected 293T cells (FIG. 10A) and C33A cells (FIG. 10B). Cells were plated at 10⁵ cells per well into 24-well plates, 24-h later they were transiently transfected with 2 μg of RNAi-3 construct DNA per well. Post-transfection 0, 24, 48, and 72-h as indicated, cell proliferation assays were performed by using Cell Titer 96® (Promega). All experiments were performed three times with triplicate samples, the results show the mean of all data and error bars indicate standard deviations. As shown in FIG. 10, it causes a reduction in cell proliferation in these two tested cells as measured by a CellTiter 96® aqueous one solution cell proliferation assay (Promega). In sum, the inventor found siRNA specific target sequence to NRIP (RNAi-3) that can efficiently knock down NRIP gene expression resulted in reduction of cell growth. It implies that the NRIP plays a pivotal role in regulating cell growth.

### EXAMPLE 9

### Methods for Diagnosis of Cancer

As the results of the examples in the invention, it believes that the NRIP antibody and RNAi-3-targeted NRIP sequence could be applied to medical diagnosis and treatment technology for the human clinical trials in cancers.

A method for diagnosis of cancers, which are related to the level of NRIP antigen expression in the patient, includes determining a level of NRIP antigen in a sample from the patient with a NRIP antibody, and comparing the level of NRIP antigen in the sample to a reference value representing a known disease or health status, whereby any elevated levels of NRIP antigen are indicative of the presence, susceptibility to, or progression of, the cancer in the patient. The level of NRIP antigen in the sample is detected and quantified using an immunoassay and/or a binding assay. For example, tissue sections of the patient are used to test with the NRIP antibody by standard immunohistochemisty assay.

Another method for diagnosis of cancer is detecting the quantity of NRIP antigen in biological fluids. An anti-NRIP antibody is bound to the wells of a microtiter plate. Tris-buffered saline or the like containing detergent plus bovine serum albumin is used to block the antibody bound microtiter well. A quantity of biological fluid that selected from cerebrospinal fluid, blood, plasma, serum, urine, sputum, saliva, urine and stool is added to the microtiter well. After incubating the biological fluid to bind the antibody, a second labeled monoclonal antibody that against the same NRIP antigen but different epitope is added to the microtiter well. Then incubate with a substrate until a significant color reaction develops to detect antibody bound materials.

### EXAMPLE 10

### A Possible Method of Treatment for Cervical Cancer

Gene silencing tools can be used both in vitro and in vivo to inhibit specific target mRNA molecules and create phenotypic changes. Although, siRNA, a novel gene-silencing tool that producing similar effect to antisense molecules, i.e. inhibition of gene expression. However, the optimism is underlined by the mode of action of siRNA, which has been reported to be 10 to 100 fold more potent in gene silencing than antisense. Therefore, siRNA molecules have not only been shown to be valuable target identification and validation tools, but have also emerged as a potential new class of therapeutics.

CaSki cell line was originally derived from cells from a metastasis in the small bowel mesentery to cervix. The cells are reported to contain an integrated human papillomavirus type 16 genome (HPV-16, about 600 copies per cell) as well as sequences related to HPV-18. It has been reported that CaSki cervical cancer cells bearing tumorigenesis in xenograft of nude mice (Kuroda et al., Br. J. Cancer, 2005, 92, 290-293). Therefore, the inventors investigated the growth inhibition of cervix carcinoma cells in vivo by NRIP blockade (Adv-RNAi-3). Initially, female nu+/nu+mice were 8 weeks old. Single cell suspension of 1.5×10⁶ of CaSki cells with viability>95% was injected subcutaneously into the flank regions of nude mice. Palpable tumors were detected about 7 days after cell injection. Tumor burden was measured with a caliper and calculated as length x width²×0.5. Secondary, the treatment was started at various times after the xenograft. 100 μl of a solution containing an active siRNA virus, rAdv-RNAi-3 (MOI˜10) specific for NRIP or the negative control of rAdv-Luc (MOI˜10) siRNA virus, is injected into the CaSki transfected mice at the tumor site. After treatment at various time points, the tumor weight, tumor size, mortality, morbidity, and histology of the subcutaneous tumors in nude mice are measured to evaluate the therapeutics effect.

Many changes and modifications in the above described embodiment of the invention can, of course, be carried out without departing from the scope thereof. Accordingly, to promote the progress in science and the useful arts, the invention is disclosed and is intended to be limited only by the scope of the appended claims.

```
<160> NUMBER OF SEQ ID NOS: 54
<210> SEQ ID NO 1
<211> LENGTH: 324
<212> TYPE: PRT
<213> ORGANISM: Homo sapiens
<400> SEQUENCE: 1
Ala Ser Arg Asn Asp Cys Thr Ile Asp Lys Ph
#e Arg Arg Lys Asn Cys
    1 5
# 10
# 15
Pro Ser Cys Arg Leu Arg Lys Cys Tyr Glu Al
#a Gly Met Thr Leu Gly
                          20
# 25
# 30
Ala Arg Lys Leu Lys Lys Leu Gly Asn Leu Ly
#s Leu Gln Glu Glu Gly
                  35
# 40
# 45
Glu Ala Ser Ser Thr Thr Ser Pro Thr Glu Gl
#u Thr Thr Gln Lys Leu
          50
# 55
# 60
Thr Val Ser His Ile Glu Gly Tyr Glu Cys Gl
#n Pro Ile Phe Leu Asn
  65
# 70
# 75
# 80
Val Leu Glu Ala Ile Glu Pro Gly Val Val Cy
#s Ala Gly His Asp Asn
                                  85
# 90
# 95
Asn Gln Pro Asp Ser Phe Ala Ala Leu Leu Se
#r Ser Leu Asn Glu Leu
                        100
# 105
# 110
Gly Glu Arg Gln Leu Val His Val Val Lys Tr
#p Ala Lys Ala Leu Pro
                115
# 120
# 125
Gly Phe Arg Asn Leu His Val Asp Asp Gln Me
#t Ala Val Ile Gln Tyr
        130
# 135
# 140
Ser Trp Met Gly Leu Met Val Phe Ala Met Gl
#y Trp Arg Ser Phe Thr
145 1
#50 1
#55 1
#60
Asn Val Asn Ser Arg Met Leu Tyr Phe Ala Pr
#o Asp Leu Val Phe Asn
                                165
# 170
# 175
Glu Tyr Arg Met His Lys Ser Arg Met Tyr Se
#r Gln Cys Val Arg Met
                        180
# 185
# 190
Arg His Leu Ser Gln Glu Phe Gly Trp Leu Gl
#n Ile Thr Pro Gln Glu
                195
# 200
# 205
Phe Leu Cys Met Lys Ala Leu Leu Leu Phe Se
#r Ile Ile Pro Val Asp
        210
# 215
# 220
Gly Leu Lys Asn Gln Lys Phe Phe Asp Glu Le
#u Arg Met Asn Tyr Ile
225 2
#30 2
#35 2
#40
Lys Glu Leu Asp Arg Ile Ile Ala Cys Lys Ar
#g Lys Asn Pro Thr Ser
                                245
# 250
# 255
Cys Ser Arg Arg Phe Tyr Gln Leu Thr Lys Le
#u Leu Asp Ser Val Gln
                        260
# 265
# 270
Pro Ile Ala Arg Glu Leu His Gln Phe Thr Ph
#e Asp Leu Leu Ile Lys
                275
# 280
# 285
Ser His Met Val Ser Val Asp Phe Pro Glu Me
#t Met Ala Glu Ile Ile
        290
# 295
# 300
Ser Val Gln Val Pro Lys Ile Leu Ser Gly Ly
#s Val Lys Pro Ile Tyr
305 3
#10 3
#15 3
#20
Phe His Thr Gln
<210> SEQ ID NO 2
<211> LENGTH: 3085
<212> TYPE: DNA
<213> ORGANISM: Homo sapiens
<400> SEQUENCE: 2
ccggtgcggc tcgggtgttg aaacgggtgt cccctccccc tcctcccctc cc
#ccacgcgg 60
tggtctcccc tcccacccgg ctcaggcaga gccatgtctc ggggtggctc ct
#acccacac 120
ctgttgtggg acgtgaggaa aaggtccctc gggctggagg acccgtcccg gc
#tgcggagt 180
cgctacctgg gaagaagaga atttatccaa agattaaaac ttgaagcaac cc
#ttaatgtg 240
catgatggtt gtgttaatac aatctgttgg aatgacactg gagaatatat tt
#tatctggc 300
tcagatgaca ccaaattagt aattagtaat ccttacagca gaaaggtttt ga
#caacaatt 360
cgttcagggc accgagcaaa catatttagt gcaaagttct taccttgtac aa
#atgataaa 420
cagattgtat cctgctctgg agatggagta atattttata ccaacgttga gc
#aagatgca 480
gaaaccaaca gacaatgcca atttacgtgt cattatggaa ctacttatga ga
#ttatgact 540
gtacccaatg acccttacac ttttctctct tgtggtgaag atggaactgt ta
#ggtggttt 600
gatacacgca tcaaaactag ctgcacaaaa gaagattgta aagatgatat tt
#taattaac 660
tgtcgacgtg ctgccacgtc tgttgctatt tgcccaccaa taccatatta cc
#ttgctgtt 720
ggttgttctg acagctcagt acgaatatat gatcggcgaa tgctgggcac aa
#gagctaca 780
gggaattatg caggtcgagg gactactgga atggttgccc gttttattcc tt
#cccatctt 840
aataataagt cctgcagagt gacatctctg tgttacagtg aagatggtca ag
#agattctc 900
gttagttact cttcagatta catatatctt tttgacccga aagatgatac ag
#cacgagaa 960
cttaaaactc cttctgcgga agagagaaga gaagagttgc gacaaccacc ag
#ttaagcgt 1020
ttgagacttc gtggtgattg gtcagatact ggacccagag caaggccgga ga
#gtgaacga 1080
gaacgagatg gagagcagag tcccaatgtg tcattgatgc agagaatgtc tg
#atatgtta 1140
tcaagatggt ttgaagaagc aagtgaggtt gcacaaagca atagaggacg ag
#gaagatct 1200
cgacccagag gtggaacaag tcaatcagat atttcaactc ttcctacggt cc
#catcaagt 1260
cctgatttgg aagtgagtga aactgcaatg gaagtagata ctccagctga ac
#aatttctt 1320
cagccttcta catcctctac aatgtcagct caggctcatt cgacatcatc tc
#ccacagaa 1380
agccctcatt ctactccttt gctatcttct ccagacagtg aacaaaggca gt
#ctgttgag 1440
gcatctggac accacacaca tcatcagtct gataacaata atgaaaagct ga
#gccccaaa 1500
ccagggacag gtgaaccagt tttaagtttg cactacagca cagaaggaac aa
#ctacaagc 1560
acaataaaac tgaactttac agatgaatgg agcagtatag catcaagttc ta
#gaggaatt 1620
gggagccatt gcaaatctga gggtcaggag gaatctttcg tcccacagag ct
#cagtgcaa 1680
ccaccagaag gagacagtga aacaaaagct cctgaagaat catcagagga tg
#tgacaaaa 1740
tatcaggaag gagtatctgc agaaaaccca gttgagaacc atatcaatat aa
#cacaatca 1800
gataagttca cagccaagcc attggattcc aactcaggag aaagaaatga cc
#tcaatctt 1860
gatcgctctt gtggggttcc agaagaatct gcttcatctg aaaaagccaa gg
#aaccagaa 1920
acttcagatc agactagcac tgagagtgct accaatgaaa ataacaccaa tc
#ctgagcct 1980
cagttccaaa cagaagccac tgggccttca gctcatgaag aaacatccac ca
#gggactct 2040
gctcttcagg acacagatga cagtgatgat gacccagtcc tgatcccagg tg
#caaggtat 2100
cgagcaggac ctggtgatag acgctctgct gttgcccgta ttcaggagtt ct
#tcagacgg 2160
agaaaagaaa ggaaagaaat ggaagaattg gatactttga acattagaag gc
#cgctagta 2220
aaaatggttt ataaaggcca tcgcaactcc aggacaatga taaaagaagc ca
#atttctgg 2280
ggtgctaact ttgtaatgag tggttctgac tgtggccaca ttttcatctg gg
#atcggcac 2340
actgctgagc atttgatgct tctggaagct gataatcatg tggtaaactg cc
#tgcagcca 2400
catccgtttg acccaatttt agcctcatct ggcatagatt atgacataaa ga
#tctggtca 2460
ccattagaag agtcaaggat ttttaaccga aaacttgctg atgaagttat aa
#ctcgaaac 2520
gaactcatgc tggaagaaac tagaaacacc attacagttc cagcctcttt ca
#tgttgagg 2580
atgttggctt cacttaatca tatccgagct gaccggttgg agggtgacag at
#cagaaggc 2640
tctggtcaag agaatgaaaa tgaggatgag gaataataaa ctctttttgg ca
#agcactta 2700
aatgttctga aatttgtata agacatttat tatatttttt tctttacaga gc
#tttagtgc 2760
aattttaagg ttatggtttt tggagttttt cccttttttt gggataacct aa
#cattggtt 2820
tggaatgatt gtgtgcatga atttgggaga ttgtataaaa caaaactagc ag
#aatgtttt 2880
taaaactttt tgccgtgtat gaggagtgct agaaaatgca aagtgcaata tt
#ttccctaa 2940
ccttcaaatg tgggagcttg gatcaatgtt gaagaataat tttcatcata gt
#gaaaatgt 3000
tggttcaaat aaatttctac acttgccatt tgcatgtttg ttgctttcta at
#taaagaaa 3060
ctggttgttt taaaaaaaaa aaaaa
#
# 3085
<210> SEQ ID NO 3
<211> LENGTH: 830
<212> TYPE: PRT
<213> ORGANISM: Homo sapiens
<400> SEQUENCE: 3
Met Ser Arg Gly Gly Ser Tyr Pro His Leu Le
#u Trp Asp Val Arg Lys
    1 5
# 10
# 15
Arg Ser Leu Gly Leu Glu Asp Pro Ser Arg Le
#u Arg Ser Arg Tyr Leu
                          20
# 25
# 30
Gly Arg Arg Glu Phe Ile Gln Arg Leu Lys Le
#u Glu Ala Thr Leu Asn
                  35
# 40
# 45
Val His Asp Gly Cys Val Asn Thr Ile Cys Tr
#p Asn Asp Thr Gly Glu
          50
# 55
# 60
Tyr Ile Leu Ser Gly Ser Asp Asp Thr Lys Le
#u Val Ile Ser Asn Pro
  65
# 70
# 75
# 80
Tyr Ser Arg Lys Val Leu Thr Thr Ile Arg Se
#r Gly His Arg Ala Asn
                                  85
# 90
# 95
Ile Phe Ser Ala Lys Phe Leu Pro Cys Thr As
#n Asp Lys Gln Ile Val
                        100
# 105
# 110
Ser Cys Ser Gly Asp Gly Val Ile Phe Tyr Th
#r Asn Val Glu Gln Asp
                115
# 120
# 125
Ala Glu Thr Asn Arg Gln Cys Gln Phe Thr Cy
#s His Tyr Gly Thr Thr
        130
# 135
# 140
Tyr Glu Ile Met Thr Val Pro Asn Asp Pro Ty
#r Thr Phe Leu Ser Cys
145 1
#50 1
#55 1
#60
Gly Glu Asp Gly Thr Val Arg Trp Phe Asp Th
#r Arg Ile Lys Thr Ser
                                165
# 170
# 175
Cys Thr Lys Glu Asp Cys Lys Asp Asp Ile Le
#u Ile Asn Cys Arg Arg
                        180
# 185
# 190
Ala Ala Thr Ser Val Ala Ile Cys Pro Pro Il
#e Pro Tyr Tyr Leu Ala
                195
# 200
# 205
Val Gly Cys Ser Asp Ser Ser Val Arg Ile Ty
#r Asp Arg Arg Met Leu
        210
# 215
# 220
Gly Thr Arg Ala Thr Gly Asn Tyr Ala Gly Ar
#g Gly Thr Thr Gly Met
225 2
#30 2
#35 2
#40
Val Ala Arg Phe Ile Pro Ser His Leu Asn As
#n Lys Ser Cys Arg Val
                                245
# 250
# 255
Thr Ser Leu Cys Tyr Ser Glu Asp Gly Gln Gl
#u Ile Leu Val Ser Tyr
                        260
# 265
# 270
Ser Ser Asp Tyr Ile Tyr Leu Phe Asp Pro Ly
#s Asp Asp Thr Ala Arg
                275
# 280
# 285
Glu Leu Lys Thr Pro Ser Ala Glu Glu Arg Ar
#g Glu Glu Leu Arg Gln
        290
# 295
# 300
Pro Pro Val Lys Arg Leu Arg Leu Arg Gly As
#p Trp Ser Asp Thr Gly
305 3
#10 3
#15 3
#20
Pro Arg Ala Arg Pro Glu Ser Glu Arg Glu Ar
#g Asp Gly Glu Gln Ser
                                325
# 330
# 335
Pro Asn Val Ser Leu Met Gln Arg Met Ser As
#p Met Leu Ser Arg Trp
                        340
# 345
# 350
Phe Glu Glu Ala Ser Glu Val Pro Asp Leu Gl
#u Val Ser Glu Thr Ala
                355
# 360
# 365
Met Glu Val Asp Thr Pro Ala Glu Gln Phe Le
#u Gln Pro Ser Thr Ser
        370
# 375
# 380
Ser Thr Met Ser Ala Gln Ala His Ser Thr Se
#r Ser Pro Thr Glu Ser
385 3
#90 3
#95 4
#00
Pro His Ser Thr Pro Leu Leu Ser Ser Pro As
#p Ser Glu Gln Arg Gln
                                405
# 410
# 415
Ser Val Glu Ala Ser Gly His His Thr His Hi
#s Gln Ser Asp Asn Asn
                        420
# 425
# 430
Asn Glu Lys Leu Ser Pro Lys Pro Gly Thr Gl
#y Glu Pro Val Leu Ser
                435
# 440
# 445
Leu His Tyr Ser Thr Glu Gly Thr Thr Thr Se
#r Thr Ile Lys Leu Asn
        450
# 455
# 460
Phe Thr Asp Glu Trp Ser Ser Ile Ala Ser Se
#r Ser Arg Gly Ile Gly
465 4
#70 4
#75 4
#80
Ser His Cys Lys Ser Glu Gly Gln Glu Glu Se
#r Phe Val Pro Gln Ser
                                485
# 490
# 495
Ser Val Gln Pro Pro Glu Gly Asp Ser Glu Th
#r Lys Ala Pro Glu Glu
                        500
# 505
# 510
Ser Ser Glu Asp Val Thr Lys Tyr Gln Glu Gl
#y Val Ser Ala Glu Asn
                515
# 520
# 525
Pro Val Glu Asn His Ile Asn Ile Thr Gln Se
#r Asp Lys Phe Thr Ala
        530
# 535
# 540
Lys Pro Leu Asp Ser Asn Ser Gly Glu Arg As
#n Asp Leu Asn Leu Asp
545 5
#50 5
#55 5
#60
Arg Ser Cys Gly Val Pro Glu Glu Ser Ala Se
#r Ser Glu Lys Ala Lys
                                565
# 570
# 575
Glu Pro Glu Thr Ser Asp Gln Thr Ser Thr Gl
#u Ser Ala Thr Asn Glu
                        580
# 585
# 590
Asn Asn Thr Asn Pro Glu Pro Gln Phe Gln Th
#r Glu Ala Thr Gly Pro
                595
# 600
# 605
Ser Ala His Glu Glu Thr Ser Thr Arg Asp Se
#r Ala Leu Gln Asp Thr
        610
# 615
# 620
Asp Asp Ser Asp Asp Asp Pro Val Leu Ile Pr
#o Gly Ala Arg Tyr Arg
625 6
#30 6
#35 6
#40
Ala Gly Pro Gly Asp Arg Arg Ser Ala Val Al
#a Arg Ile Gln Glu Phe
                                645
# 650
# 655
Phe Arg Arg Arg Lys Glu Arg Lys Glu Met Gl
#u Glu Leu Asp Thr Leu
                        660
# 665
# 670
Asn Ile Arg Arg Pro Leu Val Lys Met Val Ty
#r Lys Gly His Arg Asn
                675
# 680
# 685
Ser Arg Thr Met Ile Lys Glu Ala Asn Phe Tr
#p Gly Ala Asn Phe Val
        690
# 695
# 700
Met Ser Gly Ser Asp Cys Gly His Ile Phe Il
#e Trp Asp Arg His Thr
705 7
#10 7
#15 7
#20
Ala Glu His Leu Met Leu Leu Glu Ala Asp As
#n His Val Val Asn Cys
                                725
# 730
# 735
Leu Gln Pro His Pro Phe Asp Pro Ile Leu Al
#a Ser Ser Gly Ile Asp
                        740
# 745
# 750
Tyr Asp Ile Lys Ile Trp Ser Pro Leu Glu Gl
#u Ser Arg Ile Phe Asn
                755
# 760
# 765
Arg Lys Leu Ala Asp Glu Val Ile Thr Arg As
#n Glu Leu Met Leu Glu
        770
# 775
# 780
Glu Thr Arg Asn Thr Ile Thr Val Pro Ala Se
#r Phe Met Leu Arg Met
785 7
#90 7
#95 8
#00
Leu Ala Ser Leu Asn His Ile Arg Ala Asp Ar
#g Leu Glu Gly Asp Arg
                                805
# 810
# 815
Ser Glu Gly Ser Gly Gln Glu Asn Glu Asn Gl
#u Asp Glu Glu
                        820
# 825
# 830
<210> SEQ ID NO 4
<211> LENGTH: 8
<212> TYPE: PRT
<213> ORGANISM: Homo sapiens
<400> SEQUENCE: 4
Ser Tyr Pro His Leu Leu Trp Asp
#5
<210> SEQ ID NO 5
<211> LENGTH: 20
<212> TYPE: PRT
<213> ORGANISM: Homo sapiens
<400> SEQUENCE: 5
Phe Ile Gln Arg Leu Lys Leu Glu Ala Thr Le
#u Asn Val His Asp Gly
#5
#10
#15
Cys Val Asn Thr
                          20
<210> SEQ ID NO 6
<211> LENGTH: 8
<212> TYPE: PRT
<213> ORGANISM: Homo sapiens
<400> SEQUENCE: 6
Asp Thr Lys Leu Val Ile Ser Asn
# 5
<210> SEQ ID NO 7
<211> LENGTH: 7
<212> TYPE: PRT
<213> ORGANISM: Homo sapiens
<400> SEQUENCE: 7
Tyr Ser Arg Lys Val Leu Thr
#5
<210> SEQ ID NO 8
<211> LENGTH: 11
<212> TYPE: PRT
<213> ORGANISM: Homo sapiens
<400> SEQUENCE: 8
Ala Asn Ile Phe Ser Ala Lys Phe Leu Pro Cy
#s
#5
#10
<210> SEQ ID NO 9
<211> LENGTH: 17
<212> TYPE: PRT
<213> ORGANISM: Homo sapiens
<400> SEQUENCE: 9
Asn Asp Lys Gln Ile Val Ser Cys Ser Gly As
#p Gly Val Ile Phe Tyr
#5
#10
#15
Thr
<210> SEQ ID NO 10
<211> LENGTH: 9
<212> TYPE: PRT
<213> ORGANISM: Homo sapiens
<400> SEQUENCE: 10
Arg Gln Cys Gln Phe Thr Cys His Tyr
#5
<210> SEQ ID NO 11
<211> LENGTH: 7
<212> TYPE: PRT
<213> ORGANISM: Homo sapiens
<400> SEQUENCE: 11
Pro Tyr Thr Phe Leu Ser Cys
#5
<210> SEQ ID NO 12
<211> LENGTH: 27
<212> TYPE: PRT
<213> ORGANISM: Homo sapiens
<400> SEQUENCE: 12
Arg Ala Ala Thr Ser Val Ala Ile Cys Pro Pr
#o Ile Pro Tyr Tyr Ile
    1 5
# 10
# 15
Ala Val Gly Cys Ser Asp Ser Ser Val Arg Il
#e
                          20
# 25
<210> SEQ ID NO 13
<211> LENGTH: 8
<212> TYPE: PRT
<213> ORGANISM: Homo sapiens
<400> SEQUENCE: 13
Met Val Ala Arg Phe Ile Pro Ser
    1 5
<210> SEQ ID NO 14
<211> LENGTH: 13
<212> TYPE: PRT
<213> ORGANISM: Homo sapiens
<400> SEQUENCE: 14
Asn Asn Lys Ser Cys Arg Val Thr Ser Leu Cy
#s Tyr Ser
#5
#10
<210> SEQ ID NO 15
<211> LENGTH: 15
<212> TYPE: PRT
<213> ORGANISM: Homo sapiens
<400> SEQUENCE: 15
Gln Glu Ile Leu Val Ser Tyr Ser Ser Asp Ty
#r Ile Tyr Leu Phe
#5
#10
#15
<210> SEQ ID NO 16
<211> LENGTH: 7
<212> TYPE: PRT
<213> ORGANISM: Homo sapiens
<400> SEQUENCE: 16
Arg Gln Pro Pro Val Lys Arg
#5
<210> SEQ ID NO 17
<211> LENGTH: 16
<212> TYPE: PRT
<213> ORGANISM: Homo sapiens
<400> SEQUENCE: 17
Asp Ile Ser Thr Leu Pro Thr Val Pro Ser Se
#r Pro Asp Leu Glu Val
#5
#10
#15
<210> SEQ ID NO 18
<211> LENGTH: 7
<212> TYPE: PRT
<213> ORGANISM: Homo sapiens
<400> SEQUENCE: 18
Ala Glu Gln Phe Leu Gln Pro
#5
<210> SEQ ID NO 19
<211> LENGTH: 9
<212> TYPE: PRT
<213> ORGANISM: Homo sapiens
<400> SEQUENCE: 19
Pro His Ser Thr Pro Leu Leu Ser Ser
#5
<210> SEQ ID NO 20
<211> LENGTH: 10
<212> TYPE: PRT
<213> ORGANISM: Homo sapiens
<400> SEQUENCE: 20
Thr Gly Glu Pro Val Leu Ser Leu His Tyr
#5
#10
<210> SEQ ID NO 21
<211> LENGTH: 12
<212> TYPE: PRT
<213> ORGANISM: Homo sapiens
<400> SEQUENCE: 21
Glu Glu Ser Phe Val Pro Gln Ser Ser Val Gl
#n Pro
#5
#10
<210> SEQ ID NO 22
<211> LENGTH: 8
<212> TYPE: PRT
<213> ORGANISM: Homo sapiens
<400> SEQUENCE: 22
Asp Val Thr Lys Tyr Gln Glu Gly
#5
<210> SEQ ID NO 23
<211> LENGTH: 9
<212> TYPE: PRT
<213> ORGANISM: Homo sapiens
<400> SEQUENCE: 23
Leu Asp Arg Ser Cys Gly Val Pro Glu
#5
<210> SEQ ID NO 24
<211> LENGTH: 8
<212> TYPE: PRT
<213> ORGANISM: Homo sapiens
<400> SEQUENCE: 24
Asp Asp Pro Val Leu Ile Pro Gly
#5
<210> SEQ ID NO 25
<211> LENGTH: 7
<212> TYPE: PRT
<213> ORGANISM: Homo sapiens
<400> SEQUENCE: 25
Arg Ser Ala Val Ala Arg Ile
#5
<210> SEQ ID NO 26
<211> LENGTH: 11
<212> TYPE: PRT
<213> ORGANISM: Homo sapiens
<400> SEQUENCE: 26
Ile Arg Arg Pro Leu Val Lys Met Val Tyr Ly
#s
#5
#10
<210> SEQ ID NO 27
<211> LENGTH: 8
<212> TYPE: PRT
<213> ORGANISM: Homo sapiens
<400> SEQUENCE: 27
Ser Asp Cys Gly His Ile Phe Ile
#5
<210> SEQ ID NO 28
<211> LENGTH: 7
<212> TYPE: PRT
<213> ORGANISM: Homo sapiens
<400> SEQUENCE: 28
Thr Ala Glu His Leu Met Leu
#5
<210> SEQ ID NO 29
<211> LENGTH: 22
<212> TYPE: PRT
<213> ORGANISM: Homo sapiens
<400> SEQUENCE: 29
Glu Ala Asp Asn His Val Val Asn Cys Leu Gl
#n Pro His Pro Phe Asp
#5
#10
#15
Pro Ile Leu Ala Ser Ser
                          20
<210> SEQ ID NO 30
<211> LENGTH: 9
<212> TYPE: PRT
<213> ORGANISM: Homo sapiens
<400> SEQUENCE: 30
Ile Asp Tyr Asp Ile Lys Ile Trp Ser
#5
<210> SEQ ID NO 31
<211> LENGTH: 7
<212> TYPE: PRT
<213> ORGANISM: Homo sapiens
<400> SEQUENCE: 31
Thr Ile Thr Val Pro Ala Ser
#5
<210> SEQ ID NO 32
<211> LENGTH: 10
<212> TYPE: PRT
<213> ORGANISM: Homo sapiens
<400> SEQUENCE: 32
Met Leu Arg Met Leu Ala Ser Leu Asn His
#5
#10
<210> SEQ ID NO 33
<211> LENGTH: 19
<212> TYPE: DNA
<213> ORGANISM: Homo sapiens
<400> SEQUENCE: 33
gatggaactg ttaggtggt
#
#
# 19
<210> SEQ ID NO 34
<211> LENGTH: 19
<212> TYPE: DNA
<213> ORGANISM: Homo sapiens
<400> SEQUENCE: 34
gatggtcaag agattctcg
#
#
# 19
<210> SEQ ID NO 35
<211> LENGTH: 19
<212> TYPE: DNA
<213> ORGANISM: Homo sapiens
<400> SEQUENCE: 35
gatgatacag cacgagaac
#
#
# 19
<210> SEQ ID NO 36
<211> LENGTH: 19
<212> TYPE: DNA
<213> ORGANISM: Homo sapiens
<400> SEQUENCE: 36
gagttgcgac aaccaccag
#
#
# 19
<210> SEQ ID NO 37
<211> LENGTH: 19
<212> TYPE: DNA
<213> ORGANISM: Homo sapiens
<400> SEQUENCE: 37
caccaatcct gagcctcag
#
#
# 19
<210> SEQ ID NO 38
<211> LENGTH: 19
<212> TYPE: DNA
<213> ORGANISM: Homo sapiens
<400> SEQUENCE: 38
ctgcctgcag ccacatccg
#
#
# 19
<210> SEQ ID NO 39
<211> LENGTH: 19
<212> TYPE: DNA
<213> ORGANISM: Homo sapiens
<400> SEQUENCE: 39
tcatatccga gctgaccgg
#
#
# 19
<210> SEQ ID NO 40
<211> LENGTH: 19
<212> TYPE: DNA
<213> ORGANISM: Artificial Sequence
<220> FEATURE:
<223> OTHER INFORMATION: Primer used to amplify
#the N-terminal region of
            NRIP.
<400> SEQUENCE: 40
gacgacacgg cccgggagc
#
#
# 19
<210> SEQ ID NO 41
<211> LENGTH: 136
<212> TYPE: PRT
<213> ORGANISM: Homo sapiens
<400> SEQUENCE: 41
Met Asp Lys Ala Glu Leu Ile Pro Glu Pro Pr
#o Lys Lys Lys Arg Lys
    1 5
# 10
# 15
Val Glu Leu Gly Thr Ala Ala Asn Phe Asn Gl
#n Ser Gly Asn Ile Ala
                          20
# 25
# 30
Asp Ser Ser Leu Ser Phe Thr Phe Thr Asn Se
#r Ser Asn Gly Pro Asn
                  35
# 40
# 45
Leu Ile Thr Thr Gln Thr Asn Ser Gln Ala Le
#u Ser Gln Pro Ile Ala
          50
# 55
# 60
Ser Ser Asn Val His Asp Asn Phe Met Asn As
#n Glu Ile Thr Ala Ser
  65
# 70
# 75
# 80
Lys Ile Asp Asp Gly Asn Asn Ser Lys Pro Le
#u Ser Pro Gly Trp Thr
                                  85
# 90
# 95
Asp Gln Thr Ala Tyr Asn Ala Phe Gly Ile Th
#r Thr Gly Met Phe Asn
                        100
# 105
# 110
Thr Thr Thr Met Asp Asp Val Tyr Asn Tyr Le
#u Phe Asp Asp Glu Asp
                115
# 120
# 125
Thr Pro Pro Asn Pro Lys Lys Glu
        130
# 135
<210> SEQ ID NO 42
<211> LENGTH: 147
<212> TYPE: PRT
<213> ORGANISM: Homo sapiens
<400> SEQUENCE: 42
Met Lys Leu Leu Ser Ser Ile Glu Gln Ala Cy
#s Asp Ile Cys Arg Leu
    1 5
# 10
# 15
Lys Lys Leu Lys Cys Ser Lys Glu Lys Pro Ly
#s Cys Ala Lys Cys Leu
                          20
# 25
# 30
Lys Asn Asn Trp Glu Cys Arg Tyr Ser Pro Ly
#s Thr Lys Arg Ser Pro
                  35
# 40
# 45
Leu Thr Arg Ala His Leu Thr Glu Val Glu Se
#r Arg Leu Glu Arg Leu
          50
# 55
# 60
Glu Gln Leu Phe Leu Leu Ile Phe Pro Arg Gl
#u Asp Leu Asp Met Ile
  65
# 70
# 75
# 80
Leu Lys Met Asp Ser Leu Gln Asp Ile Lys Al
#a Leu Leu Thr Gly Leu
                                  85
# 90
# 95
Phe Val Gln Asp Asn Val Asn Lys Asp Ala Va
#l Thr Asp Arg Leu Ala
                        100
# 105
# 110
Ser Val Glu Thr Asp Met Pro Leu Thr Leu Ar
#g Gln His Arg Ile Ser
                115
# 120
# 125
Ala Thr Ser Ser Ser Glu Glu Ser Ser Asn Ly
#s Gly Gln Arg Gln Leu
        130
# 135
# 140
Thr Val Ser
145
<210> SEQ ID NO 43
<211> LENGTH: 27
<212> TYPE: DNA
<213> ORGANISM: Artificial Sequence
<220> FEATURE:
<223> OTHER INFORMATION: Primer used to amplify
#the N-terminal region of
            NRIP.
<400> SEQUENCE: 43
ccatcctaat acgactcact atagggc
#
# 27
<210> SEQ ID NO 44
<211> LENGTH: 27
<212> TYPE: DNA
<213> ORGANISM: Artificial Sequence
<220> FEATURE:
<223> OTHER INFORMATION: Primer used to amplify
#the N-terminal region of
            NRIP.
<400> SEQUENCE: 44
gaggtcattt ctttctcctg agttgga
#
# 27
<210> SEQ ID NO 45
<211> LENGTH: 23
<212> TYPE: DNA
<213> ORGANISM: Artificial Sequence
<220> FEATURE:
<223> OTHER INFORMATION: Primer used to amplify
#the N-terminal region of
            NRIP.
<400> SEQUENCE: 45
actcactata gggctcgagc ggc
#
# 23
<210> SEQ ID NO 46
<211> LENGTH: 25
<212> TYPE: DNA
<213> ORGANISM: Artificial Sequence
<220> FEATURE:
<223> OTHER INFORMATION: Primer used to amplify
#the N-terminal region of
            NRIP.
<400> SEQUENCE: 46
actggttcac ctgtccctgg tttgg
#
# 25
<210> SEQ ID NO 47
<211> LENGTH: 36
<212> TYPE: DNA
<213> ORGANISM: Artificial Sequence
<220> FEATURE:
<223> OTHER INFORMATION: Primer used to amplify
#the N-terminal region of
            NRIP.
<400> SEQUENCE: 47
tgcgaattca tgtctcgggg tggctcctac ccacac
#
# 36
<210> SEQ ID NO 48
<211> LENGTH: 39
<212> TYPE: DNA
<213> ORGANISM: Artificial Sequence
<220> FEATURE:
<223> OTHER INFORMATION: Primer used to amplify
#the N-terminal region of
            NRIP.
<400> SEQUENCE: 48
ggtgaattct tattcctcat cctcattttc attctcttg
#
# 39
<210> SEQ ID NO 49
<211> LENGTH: 39
<212> TYPE: DNA
<213> ORGANISM: Artificial Sequence
<220> FEATURE:
<223> OTHER INFORMATION: Primer used to amplify
#the N-terminal region of
            NRIP.
<400> SEQUENCE: 49
gacccgaaag acgacacggc ccgggagctg aaaactcct
#
# 39
<210> SEQ ID NO 50
<211> LENGTH: 39
<212> TYPE: DNA
<213> ORGANISM: Artificial Sequence
<220> FEATURE:
<223> OTHER INFORMATION: Primer used to amplify
#respectively 1427bp of
            the N-terminal region of NRIP, an
#d of the Beta-actin fragment.
<400> SEQUENCE: 50
aggagttttc agctcccggg ccgtgtcgtc tttcgggtc
#
# 39
<210> SEQ ID NO 51
<211> LENGTH: 27
<212> TYPE: DNA
<213> ORGANISM: Artificial Sequence
<220> FEATURE:
<223> OTHER INFORMATION: Primer used to amplify
#respectively 1427bp of
            the N-terminal region of NRIP, an
#d of the Beta-actin fragment.
<400> SEQUENCE: 51
atgtctcggg gtggctccta cccacac
#
# 27
<210> SEQ ID NO 52
<211> LENGTH: 25
<212> TYPE: DNA
<213> ORGANISM: Artificial Sequence
<220> FEATURE:
<223> OTHER INFORMATION: Primer used to amplify
#respectively 1427bp of
            the N-terminal region of NRIP, an
#d of the Beta-actin fragment.
<400> SEQUENCE: 52
actggttcac ctgtccctgg tttgg
#
# 25
<210> SEQ ID NO 53
<211> LENGTH: 21
<212> TYPE: DNA
<213> ORGANISM: Artificial Sequence
<220> FEATURE:
<223> OTHER INFORMATION: Primer used to amplify
#respectively 1427bp of
            the N-terminal region of NRIP, an
#d of the Beta-actin fragment.
<400> SEQUENCE: 53
accttcaaca ccccagccat g
#
#
#21
<210> SEQ ID NO 54
<211> LENGTH: 21
<212> TYPE: DNA
<213> ORGANISM: Artificial Sequence
<220> FEATURE:
<223> OTHER INFORMATION: Primer used to amplify
#respectively 1427bp of
            the N-terminal region of NRIP, an
#d of the Beta-actin fragment.
<400> SEQUENCE: 54
ctggaagagt gcctcagggc a
#
#
#21
```

What is claimed is:

1. A human transcriptional coactivator of steroid receptors having the base sequence of SEQ ID No: 2.

2. The human transcriptional coactivator of steroid receptors of claim 1, wherein the steroid receptors are androgen receptor and glucocorticoid receptor.

3. An isolated antibody which binds to an isolated polypeptide comprising consecutive amino acids having the sequence set forth in SEQ ID No: 3.

4. The antibody of claim 3, wherein the antibody binds to an isolated polypeptide comprising the sequence of SEQ ID No: 4, or SEQ ID No: 5, or SEQ ID No: 6, or SEQ ID No: 7, or SEQ ID No: 8, or SEQ ID No: 9, or SEQ ID No: 10, or SEQ ID No: 11, or SEQ ID No: 12, or SEQ ID No: 13, or SEQ ID No: 14, or SEQ ID No: 15, or SEQ ID No: 16, or SEQ ID No: 17, or SEQ ID No: 18, or SEQ ID No: 19, or SEQ ID No: 20, or SEQ ID No: 21, or SEQ ID No: 22, or SEQ ID No: 23, or SEQ ID No: 24, or SEQ ID No: 25, or SEQ ID No: 26, or SEQ ID No: 27, or SEQ ID No: 28, or SEQ ID No: 29, or SEQ ID No: 30, or SEQ ID No: 31, or SEQ ID No: 32.

5. The antibody of claim 3, wherein the antibody is a monoclonal antibody.

6. The antibody of claim 3, wherein the antibody is a polyclonal antibody.

7. The antibody of claim 3, wherein the antibody is detectable.

8. The antibody of claim 7, wherein the detectable antibody is labeled with a detectable marker.

9. The labeled antibody of claim 7, wherein the detectable marker is a radioactive label or a colorimetric, or a luminescent, or a fluorescent marker.

10. A method for diagnosis of cancer which related to the transcriptional coactivator of steroid receptors of claim 1 comprises:
(a) providing a test sample form patient;
(b) determining the level of an antigen of a protein that having the amino acid sequence of SEQ ID No: 3 in the sample with the antibody of claim 3;
(c) comparing the level of the antigen in the sample to a reference value representing a known disease or health status, whereby any elevated levels of the antigen are indicative of the presence, susceptibility to, or progression of, the cancer in the patient.

11. A method of claim 10, wherein the cancer which related to the transcriptional coactivator of steroid receptors of claim 1 is cervical cancer.

12. A method of claim 10, wherein the sample is a tissue section or biological fluids.

13. A method of claim 12, wherein the biological fluids are selected form the group consisting of cerebrospinal fluid, blood, plasma, serum, urine, sputum, saliva, urine and stool.

14. A RNA interference target sequence of the transcriptional coactivator of steroid receptors of claim 1.

15. The RNA interference target sequence of claim 14, wherein the sequence comprises SEQ ID No: 33, or SEQ ID No: 34, or SEQ ID No: 35, or SEQ ID No: 36, or SEQ ID No: 37, or SEQ ID No: 38, or SEQ ID No: 39.

16. The RNA interference target sequence of claim 14, wherein the sequence could be mutated by substituting alternative nucleic acids for the natural nucleic acids of the sequences.

17. A method for treatment of cancer which related to the transcriptional coactivator of steroid receptors of claim 1 comprises administering to a subject a therapeutic RNA interference of claim 14 in a sufficient amount to decrease the proliferation of the cancer cell.

18. A method of claim 17, wherein the cancer which related to the transcriptional coactivator of steroid receptors of claim 1 is cervical cancer.
